(19) United States
(12) Patent Application Publication
(10) Pub. No.: US 2019/0192678 A1
(43) Pub. Date: Jun. 27, 2019

(54) SYSTEM AND METHOD FOR THE TREATMENT OF DISEASE USING A HYPERSPECIFIC MODIFIED PROTEIN SYSTEM

(71) Applicant: Novather, Inc., Charlotte, NC (US)

(72) Inventor: Jayanth Venkata Nagendra S Batchu, Charlotte, NC (US)

(21) Appl. No.: 16/223,091

(22) Filed: Dec. 17, 2018

Related U.S. Application Data
(60) Provisional application No. 62/607,289, filed on Dec. 18, 2017.

(51) Int. Cl. A61K 47/64; C07K 14/705; A61K 35/17; C07K 16/28; A61P 35/00; A61K 47/68; A61K 47/54

(52) U.S. Cl. CPC A61K 47/6425; C07K 14/70521; A61K 35/17; A61K 47/555; A61P 35/00; A61K 47/6857; C07K 16/28

(57) ABSTRACT

A hyperspecific MP system is a system of at least 3 different types of Modified Proteins expressed on/in any cell type and operating together in a treatment, diagnostic, commercial method, or other commercial method(s). Using multiple Modified Proteins can significantly increase the specificity of cancer treatment among many others and decrease side effects of treatments.

### RELATED APPLICATION

This application claims the benefit of U.S. Provisional Application Ser. No. 62/607,289, filed Dec. 18, 2017, entitled SYSTEM AND METHOD FOR THE TREATMENT OF DISEASE USING A HYPERSPECIFIC MODIFIED PROTEIN SYSTEM, the entire disclosure of which is herein incorporated by reference.

### FIELD OF THE INVENTION

This invention relates to the field of immunology and immunotherapy and more particularly to protein expression.

### BACKGROUND OF THE INVENTION

Cancer is an abnormality in a cell's internal regulatory mechanisms that results in uncontrolled growth and reproduction of the cell. Normal cells make up tissues, and when these cells lose their ability to behave as a specified, controlled, and coordinated unit, (termed “dedifferentiation”), the defect leads to disarray among the cell population. When this occurs, a tumor begins to propagate.

In addressing a cancerous condition, the essence of many medical treatments and procedures involves the removal or destruction of the tumor tissue. Examples of significant types of treatments include the surgical removal of cancerous growths and the destruction of metastatic tumors through chemotherapy and/or radiation therapy.

Surgery often is the first step in the treatment of cancer. The objective of surgery varies. Sometimes it is used to remove as much of the evident cancerous tumor as possible, or at least to “debulk” it (remove the major bulk(s) of tumor so that there is less that needs to be treated by other techniques). Depending on the type of cancer and its location, surgery can also provide some symptomatic relief to the patient. For example, if a surgeon can remove a large portion of an expanding brain tumor, the pressure inside the skull will decrease, leading to improvement in the patient's symptoms.

However, not all tumors are amenable to surgery. Some can be located in parts of the body that render them impossible to completely excise. Examples of these would include tumors in the brainstem (a part of the brain that controls breathing) or a tumor which has grown in and around a major blood vessel. In these cases, the role of surgery is limited due to the high risk associated with tumor removal.

In some cases, surgery is not employed to debulk a tumor because it is simply not necessary. An example is Hodgkin's lymphoma, a cancer of the lymph nodes that responds very well to combinations of chemotherapy and radiation therapy. In Hodgkin's lymphoma, surgery is rarely needed to achieve cure, but almost always used to establish a diagnosis (i.e. in the form of a biopsy).

Chemotherapy is another common form of cancer treatment. Chemotherapy involves the use of medications (usually administered orally or by injection) which specifically attack rapidly dividing cells (such as those found in a tumor) throughout the body. This makes chemotherapy useful in treating cancers that have already metastasized, as well as tumors that have a high chance of spreading through the blood and lymphatic systems but are not evident beyond the primary tumor. Chemotherapy can also be used to enhance the response of localized tumors to surgery and radiation therapy. This is the case, for example, for some cancers of the head and neck.

Unfortunately, other cells in the human body that also normally divide rapidly (such as the lining of the stomach and hair) also are affected by chemotherapy. For this reason, many chemotherapy agents induce undesirable side effects such as nausea, vomiting, anemia, hair loss or other symptoms. These side effects are temporary, and there exist medications that can help alleviate many of these side effects. As knowledge in the medical arts has continued to grow, researchers have devised newer chemotherapeutic agents that are not only better at killing cancer cells, but that also result in fewer side effects for the patient.

As also discussed generally above, radiation therapy is another commonly used weapon in the fight against cancer. Ionizing radiation kills cancer by penetrating skin and intervening tissue, and damaging the DNA within the tumor cells. The radiation is delivered in different ways. The most common delivery technique involves directing a beam of radiation at the patient in a highly precise manner, focusing on the tumor. In performing this treatment, a patient lies on a table and the beam source moves around him or her, while transmitting the therapeutic radiation dose in a directed manner. The procedure lasts minutes, but can be performed daily for several weeks (depending on the type of tumor), to achieve a particular total prescribed dose. A radioisotope can be safely used to deliver local radiation for cancer treatment. A typical example of a radioisotope is I-131 for the treatment of thyroid cancer.

Another radiation method sometimes employed, called brachytherapy, involves implanting radioactive pellets (seeds) or wires in the patient's body in the region of the tumor. The implants can be temporary or permanent. For permanent implants, the radiation in the seeds decays over a period of days or weeks so that the patient is not rendered radioactive. For temporary implants, the entire dose of radiation is usually delivered in a few days, and the patient must remain in the hospital during that time, due to the need for observation and generally in view of his or her heightened radioactivity. For both types of brachytherapy, radiation is generally delivered to a very targeted area to gain local control over a cancer (as opposed to treating the whole body, as is accomplished using chemotherapy).

A number of other cancer therapies exist. Examples of such treatments include immunotherapy, monoclonal antibodies, anti-angiogenesis factors and gene therapy. A brief description of each of these relatively new treatment regimens is as follows:

Immunotherapy: There are various techniques designed to assist the patient's own immune system fight the cancer, separately from radiation or chemotherapy. Oftentimes, to achieve the goal, researchers inject the patient with a specially derived vaccine that strengthens the particular immune response needed to resist the cancer.

Monoclonal Antibodies: These are antibodies designed to attach to cancerous cells (but not normal cells) by taking advantage of differences between cancerous and non-cancerous cells in their antigenic and/or other characteristics. The antibodies can be administered to the patient alone or conjugated to various cytotoxic compounds or in radioactive form, such that the antibody preferentially targets the cancerous cells, thereby delivering the toxic agent or radioactivity to the desired cells.

Anti-Angiogenesis Factors: As cancer cells rapidly divide and tumors grow, they can soon outgrow their blood supply. To compensate for this, some tumors secrete a substance believed to help induce the growth of blood vessels in their vicinity, thus providing the cancer cells with a vascular source of nutrients. Experimental therapies have been designed to arrest the growth of blood vessels to tumors, thereby depriving them of needed sustenance.

Gene Therapy: Cancer is the product of a series of mutations that ultimately lead to the production of a cancer cell and its excessive proliferation. Cancers can be treated by introducing genes to the cancer cells that will act either to check or stop the cancer's proliferation, turn on the cell's programmed cell mechanisms to destroy the cell, enhance immune recognition of the cell, or express a pro-drug that converts to a toxic metabolite or a cytokine that inhibits tumor growth.

Another option of treatment for cancers is to employ nanoparticles that are tailored to be taken-up by the particular organ or tissue. For example, paclitaxel albumin-stabilized nanoparticle formulation for the treatment of metastatic adenocarcinoma of the pancreas. This therapy plus gemcitabine had an improved overall survival and progression-free survival in patients with metastatic adenocarcinoma of the pancreas when compared to the overall survival and progression-free survival in patients treated with gemcitabine alone. This therapy has been approved by the FDA.

Immunotherapy is a form of cancer treatment which has been recently on the rise and in some cases has sent cancer to total remission in patients. This field is being widely developed and researched and can significantly improve the prognosis for cancer patients.

It is therefore desirable to provide a new therapy employing immunotherapy approaches, as it is proving to be a promising treatment, to generate an immunotherapy based therapeutic response. This therapy would be significantly more effective if it produces mainly local effects and minimal or no systemic toxicity, or minimal or no overall side effects. This approach can also be applicable to treat or cure a wide variety of diseases involving organs and tissues in addition to cancer.

### SUMMARY OF THE INVENTION

This invention overcomes disadvantages of the prior art by providing a multiple modified protein immunotherapy system that provides a highly specific treatment option system using an interdisciplinary approach combining the fields of biotechnology, physics, medicine, biochemistry, and engineering. This usage of multiple types of Modified Proteins (MPs) can be employed to only allow the activation of killing and proliferative properties of white blood cells (and other cells) such as natural killer cells or cytotoxic T-lymphocytes when all the types of MPs are activated in an appropriate manner. This usage of multiple types of MPs can then be applied to direct white blood cells to kill cancer cells while avoiding killing healthy cells. This treatment can potentially result in minimal or no side effects.

In an embodiment, a hyperspecific Modified Protein (MP) system can include at least one protein base that can include an endodomain and a transmembrane region connected to the endodomain, and at least three ectodomains connected to the at least one protein base. The at least three ectodomains can be one or more targeting molecules. The targeting molecules can be LFA-1, CD3, CD45, CD28. antibody, aptamers, scFv(s), or affimers. The at least one endodomain can include activation molecules and inhibitory molecules. The at least one protein base can include at least three protein bases, and each of the at least three ectodomains are connected to separate protein bases. One or more of the at least three ectodomains can be connected to the same protein base.

In an embodiment, a method of treatment using a hyperspecific modified protein system can include drawing blood from a patient, modifying cells from the patient to express a modified protein system, the modified protein system having at least one modified protein base with an endodomain and a transmembrane region connected to the endodomain, and at least three ectodomains connected to the at least one modified protein base, and administering the modified cells to the patient. The method can include administering a booster. The method can include administering chemotherapy. The method can include modifying cells from the patient to express a modified protein system further comprises modifying cells from the patent to express activation molecules and inhibitory molecules.

In an embodiment, a hyperspecific MP system can include a cell and three or more different types of MPs on the cell, the three or more different types of MPs adapted to operate together to accomplish a function.

### BRIEF DESCRIPTION OF THE DRAWINGS

The invention description below refers to the accompanying drawings, of which:

FIG. 1 is a schematic view of an example of a Modified Protien (MP), according to an embodiment;

FIG. 2 is a schematic view of an exemplary hyperspecific MP system with three distinct types of MPs, according to an embodiment;

FIG. 3 is an exemplary treatment flowchart for a treatment employing hyperspecific MP system(s), according to an embodiment; and

FIG. 4 is a schematic view of an exemplary simMP with three distinct binding sites, according to an embodiment.

### DETAILED DESCRIPTION

The immune system is the body's protective system and defense against invaders and microorganisms. The immune system can be composed of white blood cells (leukocytes) and immune system tissues (such as lymph nodes) and organs (such as thymus and spleen). The major types of leukocytes are phagocytes and lymphocytes. The immune system can be divided into the innate immune system and the adaptive immune system. The innate immune system is the first line of defense and its immune response can be carried out by phagocytic cells such as neutrophils and macrophages, cytotoxic natural killer cells, and granulocytes. The adaptive immune system provides the subsequent immune response and launches a more specific attack. The adaptive immune system's response can typically be carried out by macrophages, dendritic cells, various types of T cells, and B cells.

The natural killer cells function as cytotoxic effectors and regulators of immune responses. The natural killer cells can be activated based on the relative signaling levels of activating and inhibitory receptors. The natural killer cells can eliminate stressed cells and produce a number of cytokines. Cytotoxic T lymphocytes eliminates damaged cells such as cancer cells and cells infected with viruses. They typically do this via a central pathway centered on the TCRs (T-cell receptors). The other types of white blood cells play significant roles in the immune responses as well. The activating protocols for both natural killer cells and cytotoxic T lymphocytes, as well as other cells, can be modified or controlled by a modified protein (MP). An MP is a protein that can be a mutated version of the original protein (including post-translational modifications/processing such as glycosylation) and/or can be composed of aspects/parts of other proteins (including post-translational modifications/processing such as glycosylation). By way of non-limiting example, the endodomain and transmembrane region can be based on the “natural endodomain” and the ectodomain can be modified to allow the natural killer cells and other cells to be activated based on the activity of the new ectodomain. An example of a MP is a CAR (chimeric antigen receptor) which can combine the endodomain function of a protein with a single chain variable fragment with a tuned affinity attached to a transmembrane domain. A CAR can be of any generation. With CARs or other MPs on cells such as natural killer cells or cytotoxic T lymphocytes, the activation processes and other processes of cells can be reassigned or controlled.

A modified protein (MP) can be a protein that is a mutated version of the original protein (including post-translational modifications/processing such as glycosylation) and/or can be composed of aspects/parts of other proteins (including post-translational modifications/processing such as glycosylation). MPs can be engineered via point mutations, slice overlap extension polymerase chain reactions, protein engineering techniques, and other protocols. By engineering MPs, properties such as affinity and functions can be controlled. Chimeric antigen receptors are also MPs. For example, chimeric antigen receptors can be made and these chimeric antigen receptors generally combine the endodomain function of a protein with a single chain variable fragment with a tuned affinity attached to a transmembrane domain. Fusion proteins are also MPs. MPs are not limited to, but can be typically made by splice overlap extension PCR. MPs can be engineered to be expressed in immune cells (such as T-cells and NK cells). These MPs can provide the immune cells with new functions. For example, an application of MPs is to redirect the killing and activation process of white blood cells to certain specific targets such as cancer cells. MPs can also be made through biostable interfaces. For example, one protein domain that has been biotinylated can be bound to streptavidin attached to another protein domain. This can be termed as a MP.

The present invention is a hyperspecific MP system, also referred to as a MP system hereinbelow. A hyperspecific MP system has at least three different types of MPs expressed on/in any cell operating to accomplish function(s). At least three different types of MPs take part in the hyperspecific MP system to allow more points of control. For example, using at least three CARs on T-cells or NK cells to control each cell's respective activation process would control the activation process in a more desired way and can increase the specificity of the T-cells or NK cells the hyperspecific MP system is present in. Also, a booster can be (co)administered with the hyperspecific MP system in a time appropriate manner to ensure that the hyperspecific MP system is functioning optimally as desired. The booster can improve the efficacy of the treatment. This booster can be a protein, a hormone, an interferon, or any other entity or any combination of proteins, hormones or any other entity. Also, this booster can cover any previously unconsidered factors or anything other than the hyperspecific MP system to ensure that the hyperspecific MP system can function well (such as killing only cancer cells).

The hyperspecific MP system can be expressed on the cell type of interest (for example, white blood cells) by a series of splice overlap extension PCR reactions and other associated procedures (such as transfection) employed to appropriately express desired fusion proteins on the cell type of interest (for example, white blood cells).

A non-limiting example of a method of a hyperspecific MP system(s) treatments can be as follows. Blood can be collected from the cancer patient. The cell type of interest (for example, white blood cells) from the blood sample can be modified to express the hyperspecific MP system through splice overlap extension PCR and transfection. The cell type(s) expressing the hyperspecific MP system can be administered to the patient. A chemotherapy treatment or booster can be administered to the patient. The cell type(s) expressing the hyperspecific MP system can multiply themselves or proliferate once in the blood to high volumes and/or a high volume of the cell type(s) expressing the hyperspecific MP system can be administered in the first place.

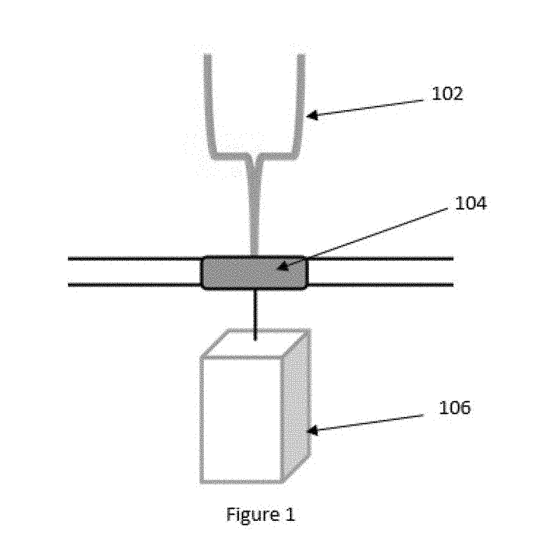

FIG. 1 describes a schematic example of a MP. An individual MP 100 can be a component in a hyperspecific MP system. The MP 100 shown in this example is a chimeric antigen receptor. Ectodomain 102 is the ectodomain region of the MP 100. The ectodomain can bind to its target cell, such as a cancer cell, and can cluster with other bound MP-target complexes, which can lead to activation of the cell. Transmembrane-domain 104 is the transmembrane region of the MP 100 and connects the ectodomain region 102 and the endodomain region 106 of the MP 100. The transmembrane region 104 also anchors the MP 100 to the membrane of consideration (such as the cell membrane). The transmembrane region 104 and the endodomain 106 can form a protein base. Endodomain 106 is the endodomain of the MP 100. The endodomain 106 can interact with other proteins in the cell and send signals. Signals that can be sent can include activation signals and/or inhibitory signals. Activation signals can include signal 1 and/or signal 2. MPs that generate activation signal(s) can be called aMPs and MPs that generate inhibitory signal(s) can be called iMPs. This endodomain can activate when clustered with other same endodomains. Clustered endodomains can recruit an enzyme or protein that can begin or end a killing process. The ectodomain can include a binding site. The binding sites can be provided by scFv(s), antibody derivatives, protein interface(s), biological interfaces, biocompatible interfaces, aptamers, etc. The ectodomain can include a binding site that can be a targeting molecule(s). a Targeting molecule can be antibiotics, glycoproteins, biocompatible interfaces, etc.

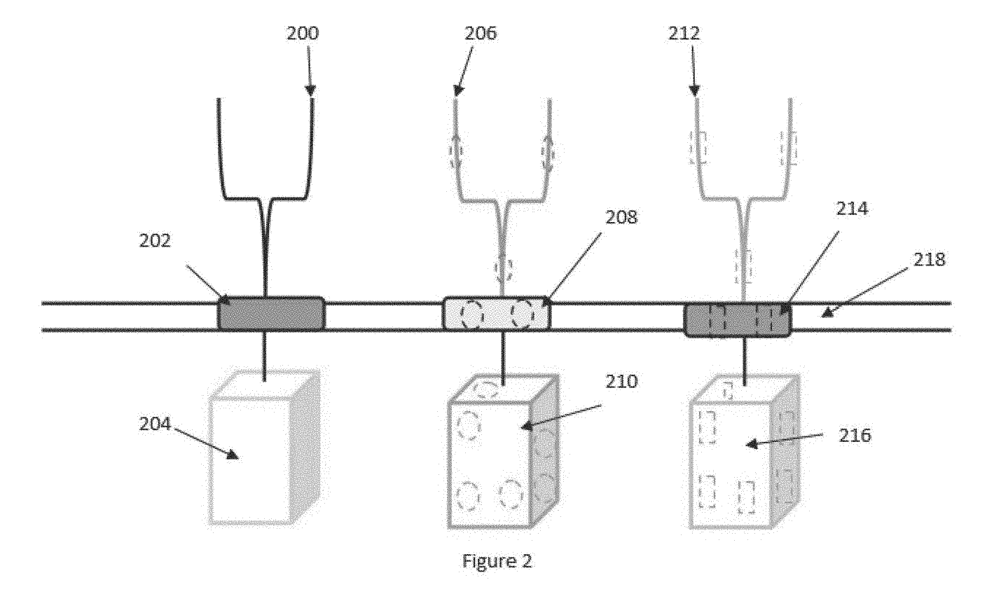

FIG. 2 describes a schematic example of the hyperspecific MP system with 3 different types of MPs. Having at least three MPs on the same surface can provide more advanced control of activation and/or inhibition signaling. This would allow for the precise targeting of target cells while also further limiting damage to non-target cells. Target cells can include cancer cells, autoimmune cells, autoreactive cells, infected cells, and anything representative or indicative of disease or diagnostic potential. Non-target cells can include healthy cells.

By way of non-limiting example, expressing two activation CARs (aCARs or CARs that generate activation signal(s)) and one inhibition CAR (iCAR or CARs that generate inhibitory signal(s)) on the same cell can provide substantially more control over the target population profile. In the past, expressing two activation CARs on the same cell has led to significant levels of toxicity, however this problem of excessive toxicity that results from expressing two activation CARs on the same cell can be solved by adding an inhibitory CAR to the system. Increasing the number of MPs/CARs increases the amount of control over signaling and cell decision making. A hyperspecific MP system 200 has at least 3 different types of MPs 210, 220, and 230, expressed on/in any cell type. In this example, all MPs are chimeric antigen receptors. In this example, membrane 218 is the cell membrane of the cell the hyperspecific MP system is expressed in, which by way of non-limiting example could be a white blood cell. In the case of FIG. 2, the ectodomains are outside of the cell, the transmembrane domains are spanning the membrane of the cell, and the endodomains are inside the cell. A first type of MP 210 can have ectodomain 212, transmembrane 214, and endodomain 216 in this example. A second type of MP 220 can have ectodomain 222, transmembrane 224, and endodomain 226 in this example. A third type of MP 230 can have ectodomain 232, transmembrane 234, and endodomain 236 in this example. All of these types of MPs 200, 210, and 220 are expressed on the same cell in this example. These three types of MPs make up the hyperspecific MP system 200 in this example. The ectodomains are typically single-chain variable fragments and can be changed to target/bind to different entities. The transmembrane domains connect the ectodomains and the endodomains while anchoring the MPs to the membrane of consideration in the cell. The endodomains of the proteins, which by way of non-limiting example can be including but not limited to LFA-1, CD3, CD45, and CD28, which can be used as the endodomains of the MPs in this example to control the killing and proliferative of white blood cells for example.

The hyperspecific MP system can be expressed on the cell type of interest by a series of splice overlap extension PCR reactions and other associated procedures (such as transfection) employed to appropriately express desired modified proteins on the cell type of interest.

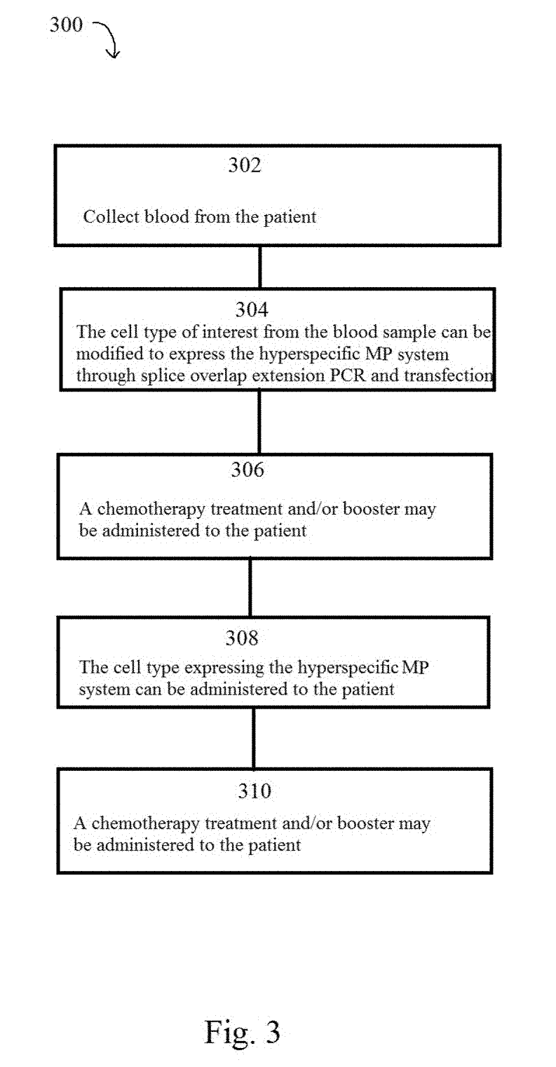

A potential treatment method employing a hyperspecific MP system(s) is described in FIG. 3. In various embodiments this treatment method 300, called the hyper specific modified protein system, can be employed with or without a booster. In an exemplary method, blood can be collected from the cancer patient at 302. At 304, the cell type of interest (for example, white blood cells) from the blood sample can be modified to express the hyperspecific MP system. This modification can be done through splice overlap extension PCR and transfection, or other methods as known in the art. Splice overlap extension PCR can be performed after modifying PCR primers in a context specific manner to overlap two or more template sequences and joining them in resulting PCR reactions and protocol steps. Splice overlap extension PCR is further explained in the article Gene Splicing by Overlap Extension: Tailor-Made Genes Using the Polymerase Chain Reaction, the entire content of which is herein incorporated by reference. Transfection can be considered to be the moving of genetic material into cells as discussed in the paper Chimeric antigen receptor T-cell therapy for solid tumors, the entire content of which is herein incorporated by reference. At 306, a chemotherapy treatment and/or booster can be administered to the patient. At 308, the cell type expressing the hyperspecific MP system can pbe administered to the patient. At 310, a chemotherapy treatment and/or booster can be administered to the patient. It should be clear that there are a variety of ways to administer the MP system treatment to a patient. By way of non-limiting example, allogenic cells could be used instead of the patient's own blood/blood cells. Other gene editing techniques such as meganucleases, CRISPR-Cas9, etc. can be employed. Cells from other sources such as cell lines can be used. Many cell types can be employed, including NK cells. Cell lines can be utilized. In various embodiments, the aforementioned steps/components can be combined in various combinations, and can be further simplified or modified to administer the MP system.

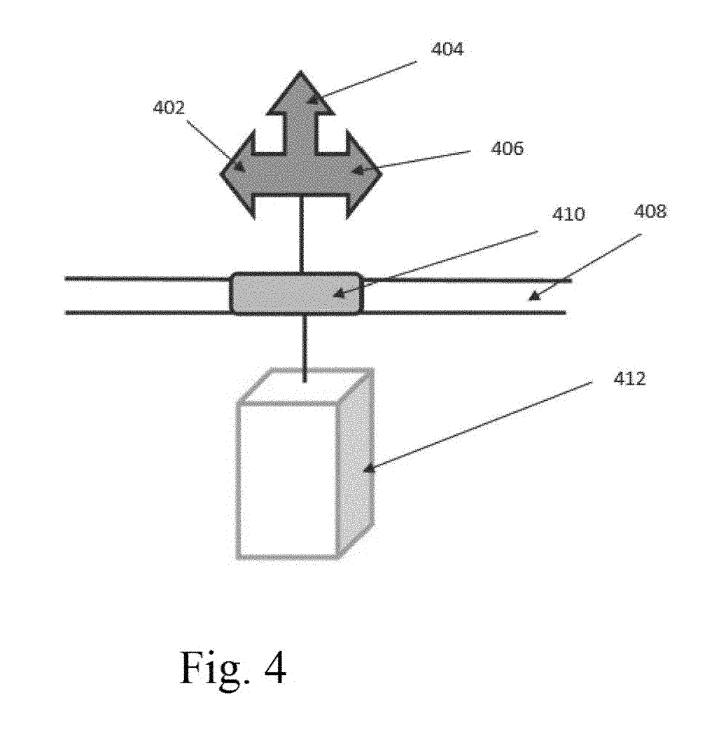

FIG. 4 shows a simMP. A simMP is a MP that has at least 3 binding sites on the ectodomain. The binding sites can be provided by scFv(s), antibody derivatives, protein interface(s), etc. A simMP can be a chimeric antigen receptor. Binding sites 402, 404, and 406 mark the binding sites of the ectodomain in FIG. 4. The binding sites may or may not be the same. In this example, membrane 408 is the membrane the simMP is expressed on/in, which by way of non-limiting example, could be a white blood cell. In the Embodiment of FIG. 4, the ectodomain is outside of the cell, the transmembrane domain is spanning the membrane of the cell, and the endodomain is inside the cell. The transmembrane domain in FIG. 4 is transmembrane 410. Endodomain 412 is the endodomain in FIG. 4. The transmembrane domain connects the ectodomain and the endodomain while anchoring the simMP to the membrane of consideration in the cell. The endodomain of the protein, which by way of non-limiting example can be including but not limited to LFA-1, CD3, CD45, and CD28, which can be used as the endodomain of the simMP in this example to control the killing and proliferative of white blood cells for example. simMPs can be used in the hyperspecific MP system. Furthermore, a simMP with n (at least 3) number of binding sites expressed on one cell can be bound to at least n (at least 3) number of proteins on another cell. simMPs can be constructed in vivo or in vitro by inserting all of the appropriate/modified components into the life form and assembling it in the life form itself to operate as desired.

A simMP can be expressed on the cell type of interest by a series of splice overlap extension PCR reactions and other associated procedures (such as transfection) employed to appropriately express desired modified proteins on the cell type of interest.

A potential treatment method utilizing simMP(s) can be very similar to the method described in FIG. 3.

The hyperspecific MP system or invention overcomes many of the disadvantages of the prior art by using an interdisciplinary approach combining the fields of biotechnology, physics, chemistry, medicine, biochemistry, and engineering to selectively activate white blood cells to kill and proliferate at the target site. The target site can be but not limited to tumor sites and metastasis sites. The use of multiple different types of MPs can have significant rises in the specificity of cancer cell killing and lead to positive results in terms of patient health. A hyperspecific MP system has at least three different types of MPs expressed on/in any cell type. This usage of multiple types of MPs can be employed to only allow the activation of killing or proliferative properties (or any function(s) as dictated by the nature of the MP of consideration) of white blood cells (or other cell types). This can be because the endodomains of the MPs expressed on the white blood cell can be the ones to activate the killing or proliferative properties after the ectodomains bind to their target in an appropriate manner. Furthermore, the hyperspecific MP system can be modified with protein switches and other modifications using various protein engineering protocols, splice overlap extension PCR, and other methods. The hyperspecific MP system can be activated by a clustering event or binding event. For example, a way the hyperspecific MP system can work is that when at least 3 different types of MPs (that make up the hyperspecific MP system in this case) are expressed in a cytotoxic t-lymphocyte or a natural killer cell, the at least 3 different types of MPs take part in clustering events while interacting with the target cell and as a result, activate the killing and proliferative properties of the cytotoxic t-lymphocyte or natural killer cell and kill the target cell. Using the hyperspecific MP system with a booster can decrease cancer treatment side effects and increase cancer treatment efficacy.

Proteins, protein fragments, or molecules that can be modified or mutated (through protein engineering techniques, splice overlap extension PCR, and other methods) to turn into MPs to be part of the hyperspecific MP system are included but not limited to LFA-1, CD3, CD45, and CD28. LFA-1, CD3, CD45, and CD28 can be used to control/regulate the killing and proliferative properties of cells and other functions. Proteins, protein fragments, or molecules that can be modified or mutated (through protein engineering techniques, splice overlap extension PCR, and other methods) to turn into MPs to be part of the hyperspecific MP system are selected based on the desired purpose. In the case of immunotherapy for cancer, the typical desired purpose is to control the killing and proliferative process of immune cells, and LFA-1, CD3, CD45, and CD28 can be selected for this purpose. The ectodomain aspect of the MP would also change based on the desired purpose. The ectodomains can change from cancer type to cancer type or from disease to disease. The MPs which make up the hyperspecific MP system can be mutated as desired. The hyperspecific MP system can be applied to any type of cell. The hyperspecific MP system can be constructed inside/in or outside/out of the target(site). The hyperspecific MP system can be constructed in vivo or in vitro by inserting all of the appropriate/modified components into the life form and assembling it in the life form itself to operate as desired.

A system in which the binding of the MPs that identify a cell as a target (aMP(s)) can generate activation signals in the cell bearing the hyperspecific MP system and/or simMP(s) and the binding of the MP(s) that identify a cell as non-target (iMP(s)) generate inhibition signals in the cell bearing the hyperspecific MP system and/or simMP(s) and the binding of the aMP(s) subsets in the effective/relevant (potentially immunological) synapse can generate enough activation signal to override the iMP(s)'s inhibition signals in the relevant/effective (potentially immunological) synapse and result in the activation of the cell engineered to bear the hyperspecific MP system and/or simMP(s). The expression and avidity of the MP(s) in the system can be controlled through promoters, protein engineering techniques, etc. Engineered MP(s) cells can activate cytotoxic and/or proliferative properties when interacting with target cells and can avoid injuring non-target cells.

The hyperspecific MP system and/or simMP(s) can be used in a balance based system, according to an embodiment. Entities overexpressed on cancer cells when compared to their expression levels on healthy cells can be targeted. Target cells can be identified by at least 3 MPs (and/or by at least 1 simMP) that recognize this overexpression. The binding of the MPs that identify a cell as a target (aMP(s)) generates activation signals in the cell bearing the hyperspecific MP system and/or simMP(s) and the binding of the MP(s) that identify a cell as non-target (iMP(s)) generate inhibition signals in the cell bearing the hyperspecific MP system and/or simMP(s). Only the binding of all of the aMP(s) subsets in the effective/relevant (potentially immunological) synapse can generate enough activation signal to override the iMP(s)'s inhibition signals in the relevant/effective (potentially immunological) synapse and result in the activation of the cell engineered to bear the hyperspecific MP system and/or simMP(s). Thus, engineered MP(s) cells can potentially activate certain properties such as cytotoxic and proliferative properties only when interacting with target cells and can avoid injuring healthy cells. If inhibition signals are more prevalent than the activation signals, then the engineered cell would not activate. This is how the balance based system can be implemented in an embodiment. The expression and avidity of the MPs can be controlled through promoters, protein engineering techniques, etc. The overall function of the engineered cell can also be modified in this manner. One of the functions of establishing a balance based system can be to modulate the potency of each signal generated by the cell it/MP system has been expressed in, in order to establish better control over the activation/kill and inhibition/not-act decision made by the cell. Another function can be to increase control over the activation/kill and inhibition/not-act decision made by the cell. In various embodiments, the MP system can be used to control a variety of processes. The MP system can be used to increase control over one or more decision making process by the cell(s). The MP system can also be used to control other processes that can including decision making. The MP system can be used to control other functions. The MP system can be used to add or delete functions.

Furthermore, a booster can be (co)administered with the hyperspecific MP system in a time appropriate manner to ensure that the hyperspecific MP system is functioning optimally as desired. This booster can be proteins, hormones, or any other entity or any combination of proteins, hormones or any other entity.

Additionally, the hyperspecific MP system can be extended into the field of diagnosis and imaging. Diagnostic and imaging compounds can be added to the hyperspecific MP systems to achieve these functions. Also, these diagnostic and imaging compounds can be released as part of the overall natural/therapeutic payload(s). Thus, these diagnostic and imaging compounds can also aid in diagnosis and imaging.

The foregoing has been a detailed description of illustrative embodiments of the invention. Various modifications and additions can be made without departing from the spirit and scope if this invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. For example, the structure of the hyperspecific MP system as described above is one of a variety of possible constructions for providing a treatment vehicle within the teachings of this invention. Also, as used herein various directional and dispositional terms such as “vertical”, “horizontal”, “up”, “down”, “bottom”, “top”, “side”, “front”, “rear”, “left”, “right”, and the like, are used only as relative conventions and not as absolute directions/dispositions with respect to a fixed coordinate system, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances of the system. Accordingly, this description is meant to be taken only by way of example, and not to otherwise limit the scope of this invention.

What is claimed is:

1. A hyperspecific Modified Protein (MP) system comprising:
at least one protein base comprising;
an endodomain; and
a transmembrane region connected to the endodomain; and
at least three ectodomains connected to the at least one protein base.

2. The MP system of claim 1, wherein the at least three ectodomains can be one or more targeting molecule(s).

3. The MP system of claim 2 wherein the targeting molecules can be LFA-1, CD3, CD45, CD28, antibody, aptamers, scFv(s), or affimers.

4. The MP system of claim 1, wherein the at least one endodomain further comprises at least one molecule selected from the group consisting of activation molecule(s) and inhibitory molecule(s).

5. The MP system of claim 1, wherein the at least one protein base comprises at least three protein bases, and each of the at least three ectodomains are connected to separate protein bases.

6. The MP system of claim 1, wherein one or more of the at least three ectodomains are connected to the same protein base.

7. A method of treatment using a hyperspecific modified protein system comprising;
drawing blood from a patient;
modifying cells from the patient to express a modified protein system, the modified protein system having at least one modified protein base with an endodomain and a transmembrane region connected to the endodomain, and at least three ectodomains connected to the at least one modified protein base; and
administering the modified cells to the patient.

8. The method of claim 7, further comprising administering a booster.

9. The method of claim 7, further comprising administering chemotherapy.

10. The method of claim 7, wherein modifying cells from the patient to express a modified protein system further comprises modifying cells from the patient to express activation molecules and inhibitory molecules.

11. A hyperspecific MP system comprising:
a cell; and
three or more different types of MPs on the cell, the three or more different types of MPS adapted to operate together to accomplish a function.
